(19) United States
(12) Patent Application Publication
(10) Pub. No.: US 2012/0184897 A1
(43) Pub. Date: Jul. 19, 2012

(54) INTEGRATED SYSTEMS AND METHODS FOR MAINTENANCE AND MANAGEMENT OF AN INTRA-ABDOMINAL GAS ENVIRONMENT DURING LAPAROSCOPIC SURGERY

(75) Inventor: Wayne L. Poll, New Albany, OH (US)

(73) Assignee: MINIMALLY INVASIVE DEVICES, LLC, Columbus, OH (US)

(21) Appl. No.: 13/185,771

(22) Filed: Jul. 19, 2011

Related U.S. Application Data
(60) Provisional application No. 61/399,863, filed on Jul. 19, 2010.

(51) Int. Cl. A61M 13/00

(52) U.S. Cl. 604/24; 604/26

(57) ABSTRACT

Air management control systems and methods maintain and manage an intra-abdominal gas environment during laparoscopic surgery. The systems and methods locate a plurality of in vivo sensors to monitor different environmental conditions within the operative space insufflated with pressurized CO2, e.g., CO2 insufflation airflow velocity, CO2 pressure, aspiration airflow velocity, and at least one of humidity level, temperature, density of smoke/particulates, odors, and sound within the operative space. The systems and methods couple the plurality of in vivo sensors to a master controller. The master controller implements pre-programmed rules to generate control commands that govern the delivery of pressurized CO2 and aspiration pressure into and out of the operative space in response, at least in part, to the different environmental conditions monitored by the in vivo sensors.

### RELATED APPLICATION

This application claims the benefit of U.S. Provisional Patent Application Ser. No. 61/399,863, filed Jul. 19, 2010, and entitled “Integrated Systems and Methods for Maintenance and Management of an Intra-Abdominal Gas Environment During Laparoscopic Surgery,” which is incorporated herein by reference.

### FIELD OF THE INVENTION

The overall purpose of this invention is an integrated system that coordinates and manages all aspects of gas flow and the intra-abdominal gas environment during laparoscopic surgery.

### BACKGROUND OF THE INVENTION

Minimally invasive surgical procedures utilizing surgical scopes, such as laparoscopes, are desirable because they often provide one or more of the following advantages: reduced blood loss; reduced post-operative patient discomfort; shortened recovery and hospitalization time; smaller incisions; and reduced exposure of internal organs to possible contaminants.

Minimally invasive surgeries permit remote visualization of a surgical site within a patient's body while the surgical procedure is being performed. During such procedures, the patient's abdominal or pelvic cavity is accessed through two or more surgically placed trocars, which enter the abdominal cavity through relatively small incisions made in the abdominal wall.

During such procedures, pressurized CO2 is used to distend the abdominal space by elevating the abdominal wall above the internal organs (pneumoperitoneum) and thereby create a sufficient working space for surgical instruments and viewing space for optics, which allow the surgeon to visualize anatomy and to perform the surgery. The CO2 is maintained at a pressure that creates an adequate working space, but does not impair the patient's physiology.

Pneumoperitoneum is created by a medical device called an insufflator, which transmits pressurized CO2 to the surgical field through insufflator tubing. The insufflator tubing enters the sterile field and connects through a connector to a surgical trocar already placed through the abdominal wall. The CO2 enters the abdominal cavity through the trocar. The insufflator monitors abdominal pressure and will moderate gas flow so as to not over-pressurize the abdominal cavity. Conventional insufflators are adapted to cycle on and off to maintain a preset and suitable pressure within the patient's body cavity. This is the sole function of the insufflator.

There are many other devices and conditions that will affect the intra-abdominal environment, all of which can affect the performance of the surgery. Currently, these other devices and conditions are not managed by the insufflator. Instead, these other devices and conditions coexist independently of each other, and sometimes lead to mutually conflicting environmental conditions affecting the surgery.

For example, surgical suction is one such independent and sometimes conflicting variable. Suction is used to remove fluids and blood during surgery, in a process called aspiration. Suction also removes air. It is not infrequent that aggressive suctioning can remove all the air from the abdominal cavity, causing a loss of the working space. This requires that surgery be interrupted until air can re-accumulate.

Another independent and sometimes conflicting variable is humidification. Currently, most insufflation is performed with dry gas that can desiccate tissues. It is believed to be advantageous to maintain a humid state in the abdominal cavity. However, separately humidification devices function independent of the insufflators. They do not function in a coordinated fashion with the rest of the air circuit. Humidification is performed without regard to the actual humidity of the abdominal cavity, and typically lasts just a small portion of the surgical procedure.

Another independent and sometimes conflicting variable is the existence of surgical smoke and plume. The use of surgical energy instruments such as harmonic scalpels and other cutting and coagulating devices generate mist, smoke, and other debris that is released into the surgical field, and which often becomes suspended throughout the expanded abdominal space. Intra-abdominal air can become contaminated from smoke and other particulates related to surgical energy instruments. These contaminants can lead to loss of optical clarity and can also, when exhausted, present a biological hazard to the surgical staff.

Laparoscope fogging is one additional independent and sometimes conflicting variable. Surgical scopes, such as laparoscopes, are commonly used also to maintain visualization of the surgical site. Conventional laparoscopes usually consist in part of a rigid or relatively rigid rod or shaft having an objective lens at one end and an eyepiece and/or integrated visual display at the other. The scope may also be connected to a remote visual display device or a video camera to record surgical procedures. The local environment within a patient's abdominal space, coupled with the release of mist, smoke, and other debris that becomes suspended throughout the expanded abdominal space, can fog the laparoscopic lens. Additionally, blood, bodily fluids, pieces of tissue, fat or other bodily material may come in contact with or even attach to the lens. As a result of these conditions, visualization through the scope can be significantly diminished. Loss of optical clarity affects surgical vision and will dirty the laparoscope lens. When this happens, the laparoscope can be removed and the lens defogged or cleaned by wiping it with a cloth, or by warming the scope tip, or by utilizing another defogging method. The need to remove the scope to defog and remove debris from the lens is inconvenient for the scope operator and the surgeon and can interrupt and undesirably prolong surgical procedures.

Fogging and debris deposition on the lens can be also reduced by devices that produce a flow of dry CO2 over the lens of the laparoscope. Currently, the flow of dry CO2 for defogging the laparoscope takes place independent of insufflator control.

Another independent and sometimes conflicting variable is the physical properties of the insufflation tubing itself. Most insufflation tubing is not sized and configured to transmit high gas flows currently available with modern insufflators. There are choke points within the tubing and at the trocar connectors, so that optimal flow cannot be achieved. Low flow insufflation frustrates efforts to maintain a working space.

All current surgical tools that affect the gas flow and intra-abdominal gas environment during laparoscopic surgery do so independent of each other, without coordination, and, as a result, their individual and combined effects are managed in a haphazard, non-optimized fashion by the operating room (OR) team.

### SUMMARY OF THE INVENTION

The invention provides integrated air management systems and methods for maintaining and managing the gas flow and intra-abdominal gas environment during laparoscopic surgery. The systems and methods maintain and manage all aspects of airflow, suction, humidity, clarity, and intra-abdominal environmental gas conditions. The systems and methods connect to the operative space through sterile tubing optimized to manage affects and capabilities.

The systems and methods make possible the achievement of one or more of the following operational objectives:

(1) A separate tube or cable coupled to in vivo sensors record continuously pressure, humidity, sound, and particulate matter in the operative space.

(2) Surgical suction is governed relative to intra-abdominal pressure in response to real time sensing so that suction cannot empty the cavity.

(3) Air flow is cycled continuously in response to real time sensing to remove particulate matter and smoke.

(4) Humidity is managed in response to real time sensing to maintain relative humidity above a set point

(5) Supply tubing and connectors can be optimized to deliver truly high flow insufflations.

(6) Dry air is placed over the lens through an add-on accessory (e.g., a FloShield® Device, available from Minimally Invasive Devices, Inc.) in a continuous fashion.

(7) Sound monitoring can increase gas flow in response to sound detection of the use of harmonic scalpel.

The systems and methods provide a more consistent surgical space, more efficient suction, less tissue damage through gas humidification, greater clarity of the surgical view, and a safer gas environment for patients and the surgical staff.

In one embodiment, the systems and methods locate a plurality of in vivo sensors to monitor different environmental conditions within the operative space insufflated with pressurized CO2, e.g., CO2 insufflation airflow velocity, CO2 pressure, aspiration airflow velocity, and at least one of humidity level, temperature, density of smoke/particulates, odors, and sound within the operative space. The systems and methods couple the plurality of in vivo sensors to a master controller. The master controller implements pre-programmed rules to generate control commands that govern the delivery of pressurized CO2 and aspiration pressure into and out of the operative space in response, at least in part, to the different environmental conditions monitored by the in vivo sensors.

In one embodiment, the systems and method provide an operator interface coupled to the master controller for the operator to input desired control variables and thresholds for the pre-programmed rules.

In one embodiment, the control commands of the master controller, e.g., govern operation of a heater/humidifier unit for the pressurized CO2, and/or govern operation of an aspirated air scrubbing unit, and/or govern operation of a view optimizing unit associated with a laparoscopic lens located within the operative space, according to the pre-programmed rules in response, at least in part, to the different environmental conditions monitored by the in vivo sensors.

In one embodiment, the plurality of in vivo sensors includes a sensor to monitor optical clarity of an image received by a laparoscopic lens located within the operative space.

In one embodiment, the preprogrammed rules compare sensed environment conditions to specified upper and lower thresholds and generate control commands to maintain the sensed conditions within a range bounded by the thresholds.

In one embodiment, the preprogrammed rules compare one sensed environmental condition to another sensed environmental condition and generate control commands to maintain a prescribed balance among different sensed environmental conditions.

In one embodiment, the pre-programmed rules of the master controller derive closed loop control commands for airflow devices having different functions affecting the environmental conditions within the operative space.

In one embodiment, the preprogrammed rules derive control commands that are proportional to sensed absolute deviations from control threshold(s), and/or derive control commands that are based upon changes in sensed deviations from control threshold(s) over time, and/or derive control commands that are based upon a rate of changes in sensed deviations over time.

In one embodiment, the preprogrammed rules compare changes in sensed deviations from control threshold(s) over time in one sensed environmental condition to changes in sensed deviations from control threshold(s) over time in another sensed environmental condition and generate control commands that maintain a prescribed balance among the different sensed environmental conditions.

In one embodiment, the pre-programmed rules take into account, at least in part, the physical dimensions and/or properties, and/or orientation of insufflator tubing that delivers the pressurized CO2.

Other objects, advantages, and embodiments of the invention are set forth in part in the description which follows, and in part, will be obvious from this description, or may be learned from the practice of the invention.

### BRIEF DESCRIPTION OF THE DRAWINGS

FIG. 1 is a perspective view of an operating room set up to perform a laparoscopic surgical procedure, including the presence of an air management control console that embodies the technical features described herein.

FIG. 2 is a somewhat schematic view of the air management control console shown in FIG. 1.

FIG. 3 is a somewhat schematic view of the array of sensors that are coupled by tubing to the air management control console shown in FIG. 1.

FIG. 4 is a view of a kit packaging the disposable, single-use components that are sized and configured to be used in association with the air management control console shown in FIG. 1.

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

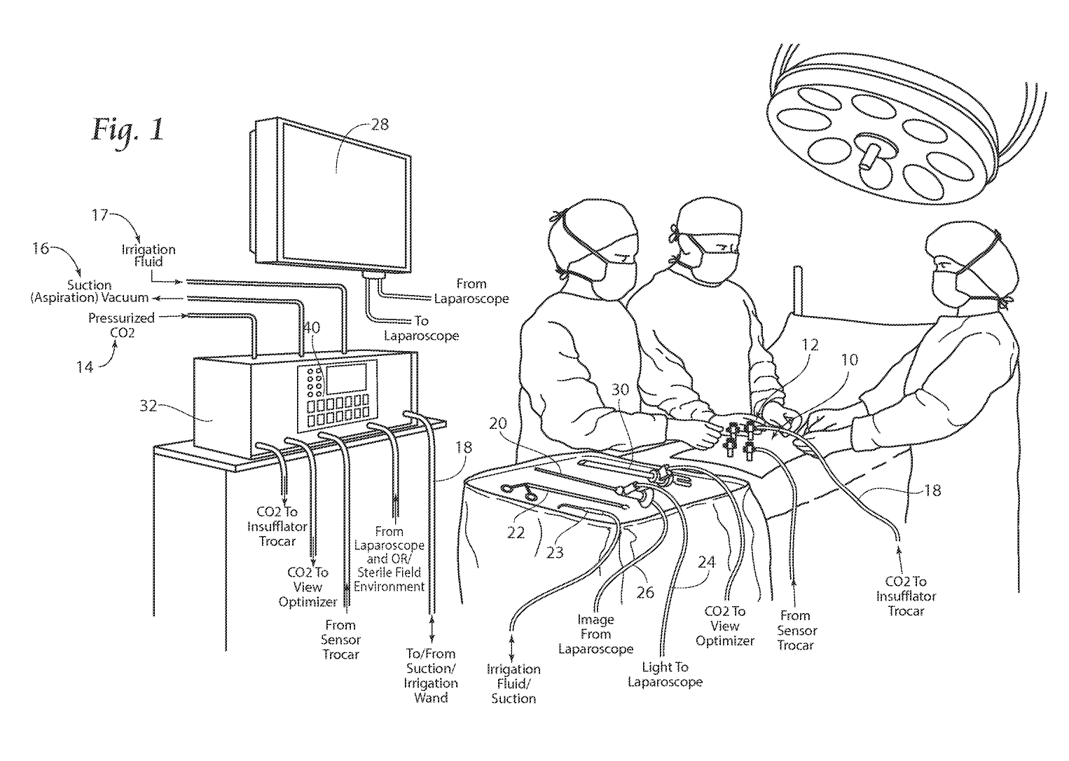

FIG. 1 shows a patient draped on a sterile field 10 for a minimally invasive, intra-abdominal surgical procedure. The OR team has made in conventional fashion incisions to insert several trocars 12 into the abdominal cavity through the abdominal wall on the sterile field. The trocars 12 provide access to a targeted operative space within the abdominal cavity.

Off the sterile field, a source of pressurized CO2 14 is set up for use during the operating procedure. Also off the sterile field, a vacuum (aspiration suction) source 16 is set up for use during the operating procedure, as is an irrigation fluid source 17.

In this arrangement, one or more of the trocars 12 can include connectors to be coupled to the source of pressurized insufflation CO2 via a length of tubing 18. The trocars 12 include lumens that convey the insufflation CO2 to the operative space.

The trocars 12 can also include lumens for introducing into the operative space other various surgical tools and instruments that aid the surgical procedures, such as, e.g., a visualization laparoscope(s) 20; devices 22 with grasping, dissecting, suturing, and/or cutting capabilities; and an irrigation/suction wand 23, which can be switched between operation in one mode to convey irrigation fluid into the operative space and another mode to aspirate by suction fluid, particulates, and smoke from the operative space. Also present on the sterile field are a light cable 24 (which directs light through the laparoscope to illuminate the operative space) and a camera cable 26 (which takes the image from the laparoscope and displays it on monitors 28 in the OR).

Other instruments may also be present on the sterile field. For example, a view optimizing assembly 30 may be provided for use in association with the laparoscope. An exemplary view optimizing assembly is a FloShield® Device available from Minimally Invasive Devices, Inc.

The FloShield® view optimizing assembly comprises a multi-lumen sheath, which mounts over the shaft of the laparoscope. The sheath includes a connector to couple to a source of anhydrous carbon dioxide (CO2). The sheath includes at its distal end a deflector assembly that is sized and configured to direct the CO2 in a prescribed flow path and flow velocity continuously across the laparoscopic lens to defog and clean the laparoscopic lens.

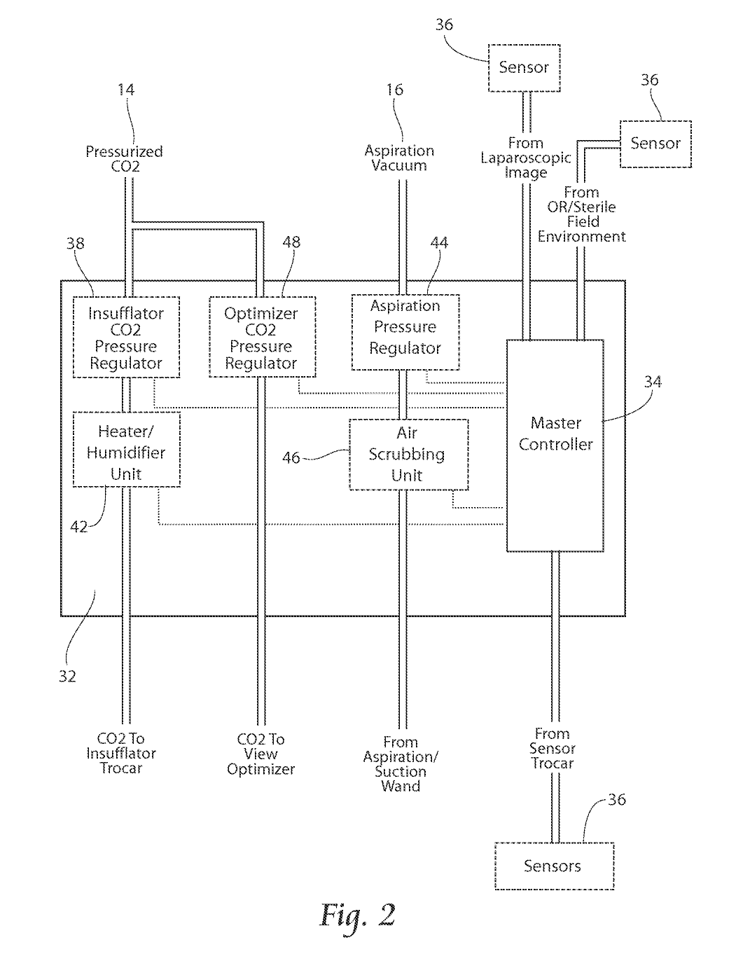

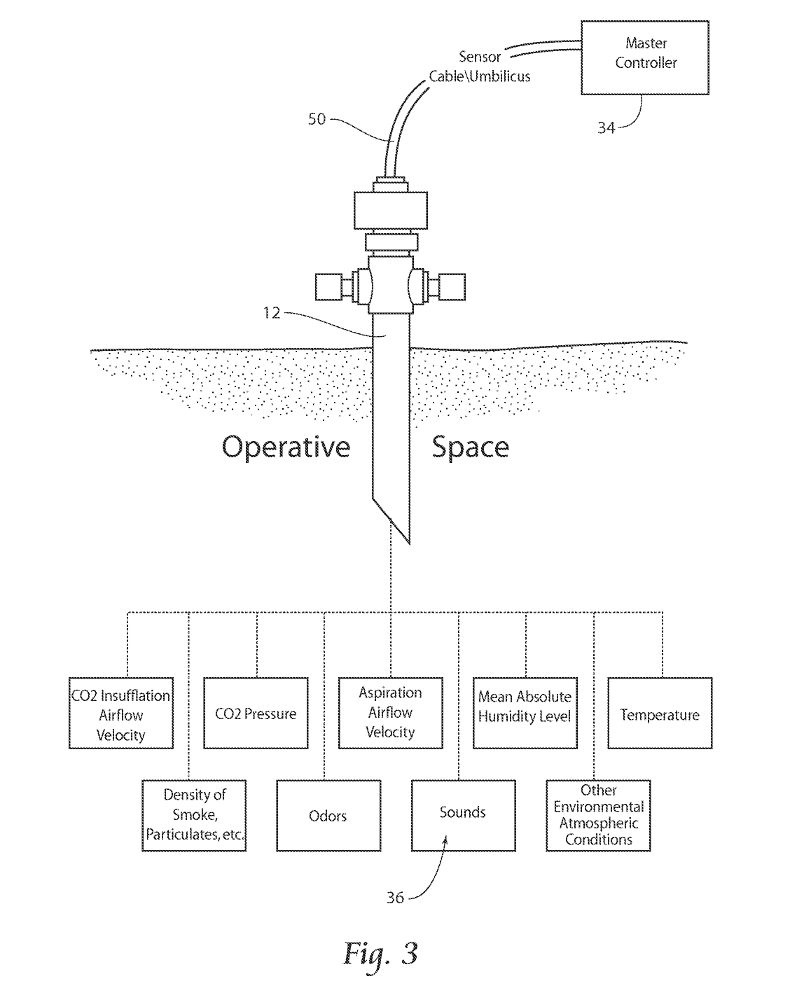

According to the invention (see FIG. 1), an air management control console 32 is set up off the sterile field. As will be described in greater detail (see FIG. 2), the air management control console 32 includes a master controller 34 that couples an array of airflow devices having different functions affecting the environmental conditions within the operative space with an array of sensors 36 (see FIG. 3) that monitor different environmental conditions within the operative space. The master controller operates the airflow devices according to pre-programmed rules (executing prescribed control algorithms) in response to environmental conditions monitored by the sensors. Desirable, a caregiver can also directly input to the master controller 34 desired control variables and thresholds through an operator interface 40 on the air management console 32. In this fashion, the master controller manages the functions of airflow, suction, humidity, optical clarity, and intra-abdominal environmental gas conditions in a coordinated manner, to optimize environmental conditions both within the operative space and in the OR.

The source of pressurized CO2 14 and the vacuum source 16 are coupled to the air management control console 32 (as FIG. 1 shows). The air management control console couples the source of pressurized CO2 to a designated insufflation trocar on the sterile field via insufflator tubing. The master controller governs the delivery of pressurized CO2 into the operative space by operating an insufflator CO2 pressure regulator 38 integrated into the control console 32 (see FIG. 2) according to the pre-programmed rules (and caregiver input) in a controlled and coordinated fashion in response, at least in part, to environmental conditions monitored by one or more of the sensors 36, as will be described in greater detail later. The controlled and coordinated delivery of pressurized CO2 generates an optimal pneumoperitoneum within the operative space that accommodates manipulation of the laparoscopic surgical instruments and optics. The master controller 34 maintains the optimal pneumoperitoneum in coordination with other environmental conditions also monitored by the sensors 36.

Desirably, the pre-programmed rules executed by the master controller take into account, at least in part, the physical dimensions, properties, and orientation of the insufflator tubing itself. The pre-programmed rules establish and maintain a maximum airflow velocity of the CO2 in the insufflator tubing, while also taking into account other environmental conditions monitored by the sensors, to provide high flow insufflation in a controlled and coordinated manner, in coordination with other environmental conditions monitored by the sensors.

Typically, in laparoscopic surgery, CO2 is insufflated at room temperature, with a relative humidity approaching 0%. To minimize the potential detrimental effects caused by desiccation, the air management control console 32 desirably includes an integrated heater/humidification unit 42 (see FIG. 2). The master controller 34 selectively places the heater/humidification unit 42 into communication with the insufflations airflow, to heat and humidify the insufflations airflow in the operative space according to the pre-programmed rules (and caregiver input) in response, at least in part, to environmental conditions monitored by the sensors 36 in a controlled and coordinated fashion, as will be described in greater detail later. The controlled and coordinated introduction of heat and water vapor into insufflation airflow maintains optimized mean absolute humidity and temperature conditions in the operating field, in coordination with other environmental conditions monitored by the sensors.

As FIG. 1 shows, the air management control console 32 couples the source of suction vacuum 16 (i.e., aspiration) and the source of irrigation fluid 17 to the irrigation/suction wand 23 via irrigation/suction tubing. The master controller 34 governs the removal of smoke, particulates, aerosolized pathogens, odors, chemical toxins, and other undesired agents from the operative space by operating an aspiration pressure regulator 44 integrated into the control console 32 (see FIG. 2). The master controller 34 operates the aspiration pressure regulator 44 according to the pre-programmed rules in response, at least in part, to environmental conditions monitored by the sensors in a controlled and coordinated fashion, as will be described in greater detail later, in coordination with other environmental conditions monitored by the sensors, to maintained optimized environmental and viewing conditions within the operative space.

Desirably, the air management control console 32 includes an integrated air scrubbing unit 46 (see FIG. 2) that traps smoke, particulates, aerosolized pathogens, odors, chemical toxins, and other undesired agents in the aspirated airflow drawn from the operative space, to prevent their reentry into the operative space and entry into the OR. For example, the air scrubbing unit 46 can include a filter media sized and configured to beneficially remove, e.g., airborne particles, smoke, pathogens, and toxins from the aspirated airflow. The filter media can comprise, e.g., at least one layer of an ultra low particulate air (ULPA) filtration material and/or a high efficiency particular air (HEPA) filtration material to remove a high percentage (e.g., 99+%) of airborne particles from the airflow. The filter media can comprise, in addition to the ULPA and/or HEPA filtration material, at least one layer of a material that absorbs smoke, odors and chemical toxins from the airflow. The layer can be formed by or incorporate, e.g., carbon or charcoal based material, or a diatomaceous earth material, or other odor removing or reducing agents.

As FIG. 1 shows, the air management control console 32 also desirably couples the source of pressurized CO2 14 to the view optimizing assembly 30 for the laparoscope 20 via view optimizing tubing. The master controller governs the delivery of pressurized CO2 to the view optimizing assembly by operating an optimizer CO2 pressure regulator 48 integrated into the control console 32 (as FIG. 2 shows). The master controller 34 operates the optimizer CO2 pressure regulator 48 according to the pre-programmed rules in response, at least in part, to environmental conditions monitored by the sensors 36 in a controlled and coordinated fashion, as will be described in greater detail later. The controlled and coordinated delivery of pressurized CO2 establishes a desired flow path and flow velocity of CO2 continuously across the laparoscopic lens to prevent fogging, in coordination with other environmental conditions monitored by the sensors. The desired flow path and flow velocity of CO2 also desirably serves to deflect smoke and surgical debris away from the laparoscopic lens during surgery.

As before stated (see FIG. 3), the master controller 34 communicates with an array of sensors 36 that monitor different environmental conditions within the operative space. The nature and type of sensors 36 can vary according to the desired control functions of the master controller 34. For example, as FIG. 3 shows, sensors 36 can be provided that communicate with the operative space to sense (i) CO2 insufflation airflow velocity entering the operative space; (ii) CO2 pressure within the operative space; (iii) aspiration airflow velocity, (iv) mean absolute humidity levels in the operative space; (v) temperature in the operative space; (vi) density of smoke, particulates, aerosolized pathogens, chemical toxins, and other undesired agents in the operative space; (vii) odors in the operative space; (viii) sound in the operative space (e.g., operation of a harmonic scalpel); and/or (ix) other intra-abdominal environmental atmospheric conditions in the operative space. Sensors can also be provided to monitor the optical clarity of the image received through the laparoscopic lens. As FIG. 2 shows, sensors 36 can also be provided to monitor optical clarity of the laparoscopic image and/or external environmental atmospheric conditions in the sterile field and/or OR.

The sensor outputs can be generated at specified intervals of time, which need not be the same time interval for each sensor. For example, the CO2 insufflation airflow velocity can be sensed at shorter time period than, e.g., temperature in the operative space.

The periodic sensor outputs are inputted to the master controller. The output of the array of sensors can be bundled in a single sensing cable 50 (see FIG. 3) that is coupled to the master controller 34 within the air management control console 32. The outputs of intra-abdominal sensors can be passed through a single sensor trocar 12 on the operating field, as FIG. 3 shows.

The master controller 34 processes the inputs according to preprogrammed rules (and caregiver inputs) to derive closed loop control commands for the array of airflow devices having different functions affecting the environmental conditions within the operative space. The preprogrammed rules of the master controller 34 can, e.g., compare the sensed output to specified upper and lower thresholds, and generate control commands that vary a given operating condition to maintain the sensed conditions within the range bounded by the thresholds. The preprogrammed rules of the master controller 34 can also compare one sensed condition to another sensed condition, and to generate control commands that are coordinated to maintain a prescribed balance among different sensed conditions. For example, the master controller 34 can employ a logic table that can dictate a selection of a corrective action according to a preprogrammed rule IF X AND Y THEN Z: e.g., IF the sensed insufflation CO2 pressure is above the minimum threshold (X) and the sensed density of smoke in the operative space is above the targeted maximum threshold (Y), THEN increase the aspiration airflow velocity (Z). In this example, if the sensed insufflation CO2 pressure was not above the desired threshold (X), the aspiration airflow velocity would not be increased, as it could lead to a collapse of pneumoperitoneum within the operative space. This is one example of how the master controller 34 can not only control, but also coordinate the processing of multiple independent and sometimes mutually conflicting variables affecting the intra-abdominal operative field.

The preprogrammed rules can provide control commands that are proportional to sensed absolute deviations from control threshold(s). Alternatively, the preprogrammed rules can provide integral or derivative control commands that are based upon the changes in the deviations over time (increasing? or decreasing?) as well as the rate of the changes in the deviations (i.e., by sensing whether the deviations are getting larger or smaller over time and by how much). The preprogrammed rules of the master controller 34 can also compare the changes over time in one sensed condition to the changes in time to another sensed condition, and to generate proportional, integral or derivative control commands that are coordinated to maintain a prescribed balance among the different sensed conditions, as before described in relation to the IF X AND Y THEN Z logic rule. Other logic rules can be applied: e.g., IF X OR Y THEN Z, or IF X AND NOT Y THEN Z.

In this way, the control commands generated by the preprogrammed rules of the master controller 34 can integrate and coordinate a host of airflow control functions, such as insufflation control (e.g., CO2 pressure and/or CO2 velocity), suction control (e.g., pressure, and/or velocity, and/or clarity/content of aspirated airflow and/or operation of cutting instruments), humidification control (e.g., mean absolute humidity and/or temperature), image clarity control (e.g., airflow velocity to the view optimizing assembly, and/or visual clarity of the image through the laparoscopic lens), air quality control of the operating field and/or the OR, and/or other environmental management control internal and external to the operative space.

The air management control console 32 as described is an intelligent system that manages all aspects of airflow, suction, humidity, temperature, clarity, and intra-abdominal environmental gas conditions. The air management control console 32 connects to the operative space through sterile tubing. The master controller 34 receives input from an array of sensors present in the operative space or in the OR. The master controller 34 executes pre-programmed rules to balance and optimize the sometimes conflicting effects and capabilities of different airflow devices.

For example, by sensing continuously pressure, humidity, sound, and particulate matter in the operative space, (i) surgical suction (aspiration) can be governed relative to intra-abdominal pressure so that suction cannot empty the cavity; (ii) CO2 air flow can be cycled continuously in a metered way to remove particulate matter and smoke; (iii) humidity and/or temperature can be managed in real time to maintain relative humidity and/or temperature above a set point; (iv) supply tubing and connectors can be optimized to deliver truly high flow insufflation; (vi) dry air can be placed over the lens through an add-on accessory (FloShield®, Minimally Invasive Devices, Inc.) in a continuous fashion; and (vii) sound monitoring can increase gas flow in response to the operation of a harmonic scalpel.

If desired, all or some of the sensed variables can be displayed on the operator interface 40, and visual or audible alarms can be activated by the master controller 34 to alert the caregiver to out of bounds conditions.

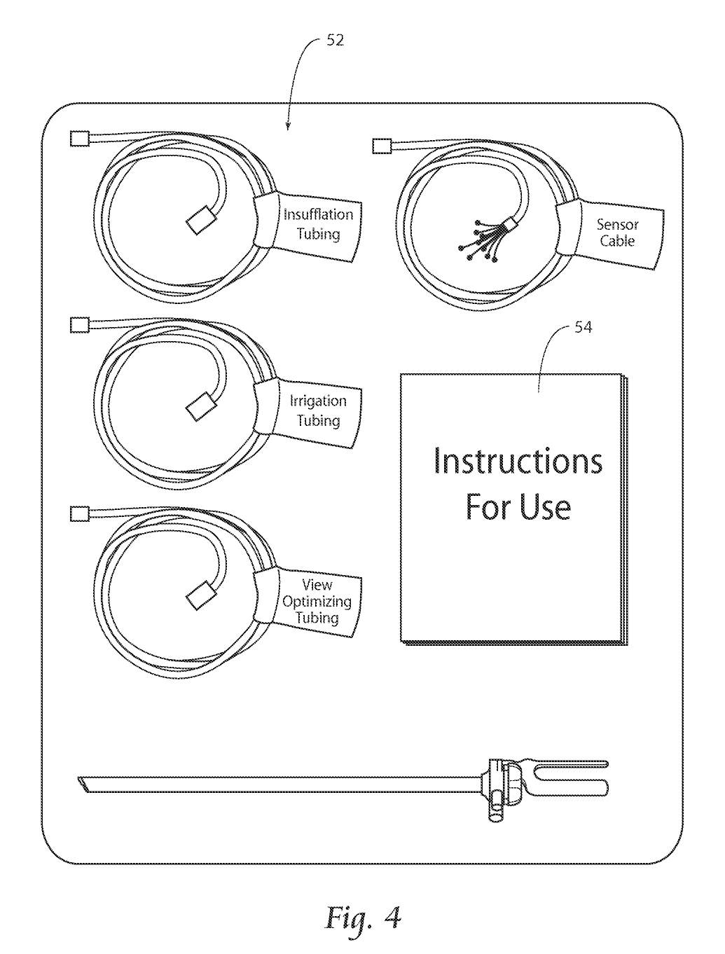

As FIG. 4 shows, the disposable, single-use components that are sized and configured to be used in association with the air management control console 32 can be packaged prior to use in a kit 52. For example, the kit can contain (i) the insufflations tubing (with connectors for coupling to the air management control console and the insufflation trocar); (ii) the suction/irrigation tubing (with connectors for coupling to the air management control console and the suction/irrigation wand); (iii) the view optimizing assembly, including the view optimizing tubing (with connectors for coupling to the air management control console and the view optimizing assembly); and (iv) the array of sensors with sensor tubing for coupling the array to sensors to the air management control console. The kit can include “Instruction for Use” 54, directing the caregiver how to make the connections among the single-use components and the air management control console, and to operate the air management control console in the manner described herein.

The technical features of the air management control console 32 can provide a more consistent surgical space, more efficient suction, less tissue damage through gas humidification, greater clarity of the surgical view, and a safer gas environment for patients and the surgical staff.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. An air management control system for maintenance and management of an intra-abdominal gas environment during laparoscopic surgery comprising
a source of pressurized CO2 for insufflating an intra-abdominal operative space,
a source of vacuum for aspirating the intra-abdominal operative space,
a plurality of in vivo sensors sized and configured to monitor different environmental conditions within the operative space insufflated with pressurized CO2, the different environmental conditions including CO2 insufflation airflow velocity, CO2 pressure, aspiration airflow velocity, and at least one of humidity level, temperature, density of smoke/particulates, odors, and sound within the operative space, and
a master controller coupled to the source of pressurized CO2 and the plurality of in vivo sensors, the master controller being conditioned to generate control commands that govern the delivery of pressurized CO2 and aspiration pressure into and out of the operative space according to pre-programmed rules in response, at least in part, to the different environmental conditions monitored by the in vivo sensors.

2. A system according to claim 1
further including an operator interface coupled to the master controller for the operator to input desired control variables and thresholds for the pre-programmed rules.

3. A system according to claim 1
further including a heater humidifier unit coupled to the source of pressurized CO2, and
wherein the control commands of the master controller govern operation of the heater/humidifier unit according to the pre-programmed rules in response, at least in part, to the different environmental conditions monitored by the in vivo sensors.

4. A system according to claim 1
further including an air scrubbing unit coupled to the source of vacuum, and
wherein the control commands of the master controller govern operation of the air scrubbing unit according to the pre-programmed rules in response, at least in part, to the different environmental conditions monitored by the in vivo sensors.

5. A system according to claim 1
further including a view optimizing unit to direct dry air across a laparoscopic lens located within the operative space to defog and clean the laparoscopic lens, and
wherein the control commands of the master controller govern operation of the view optimizing unit according to the pre-programmed rules in response, at least in part, to the different environmental conditions monitored by the in vivo sensors.

6. A system according to claim 1
wherein the plurality of in vivo sensors includes a sensor to monitor optical clarity of an image received by a laparoscopic lens located within the operative space.

7. A system according to claim 1
wherein at least some of the plurality of in vivo sensors are bundled in a single sensing cable coupled to the master controller.

8. A system according to claim 1
wherein the preprogrammed rules compare sensed environment conditions to specified upper and lower thresholds and generate control commands to maintain the sensed conditions within a range bounded by the thresholds.

9. A system according to claim 1
wherein the preprogrammed rules compare one sensed environmental condition to another sensed environmental condition and generate control commands to maintain a prescribed balance among different sensed environmental conditions.

10. A system according to claim 1
wherein the pre-programmed rules of the master controller derive closed loop control commands for airflow devices having different functions affecting the environmental conditions within the operative space.

11. A system according to claim 1
wherein the preprogrammed rules derive control commands that are proportional to sensed absolute deviations from control threshold(s).

12. A system according to claim 1
wherein the preprogrammed rules derive control commands that are based upon changes in sensed deviations from control threshold(s) over time.

13. A system according to claim 1
wherein the preprogrammed rules derive control commands that are based upon a rate of changes in sensed deviations over time.

14. A system according to claim 1
wherein the preprogrammed rules compare changes in sensed deviations from control threshold(s) over time in one sensed environmental condition to changes in sensed deviations from control threshold(s) over time in another sensed environmental condition and to generate control commands that maintain a prescribed balance among the different sensed environmental conditions.

15. A system according to claim 1
wherein the source of pressurized CO2 is coupled to the intra-abdominal operative space by insufflation tubing, and
wherein the pre-programmed rules take into account, at least in part, the physical dimensions and/or properties, and/or orientation of the insufflator tubing.

16. A system according to claim 1
wherein the master controller is housed within a single console.

17. An air management control method for maintenance and management of an intra-abdominal gas environment during laparoscopic surgery comprising
locating a plurality of in vivo sensors to monitor different environmental conditions within an operative space insufflated with pressurized CO2, the different environmental conditions including CO2 insufflation airflow velocity, CO2 pressure, aspiration airflow velocity, and at least one of humidity level, temperature, density of smoke/particulates, odors, and sound within the operative space, and
coupling the plurality of in vivo sensors to a master controller that includes pre-programmed rules to generate control commands that govern the delivery of pressurized CO2 and aspiration pressure into and out of the operative space in response, at least in part, to the different environmental conditions monitored by the in vivo sensors.

18. A method according to claim 17
providing an operator interface coupled to the master controller for the operator to input desired control variables and thresholds for the pre-programmed rules.

19. A method according to claim 17
wherein the control commands of the master controller govern operation of a heater/humidifier unit for the pressurized CO2 in response, at least in part, to the different environmental conditions monitored by the in vivo sensors.

20. A method according to claim 17
wherein the control commands of the master controller govern operation of an aspirated air scrubbing unit according to the pre-programmed rules in response, at least in part, to the different environmental conditions monitored by the in vivo sensors.

21. A method according to claim 17
wherein the control commands of the master controller govern operation of a view optimizing unit associated with a laparoscopic lens located within the operative space according to the pre-programmed rules in response, at least in part, to the different environmental conditions monitored by the in vivo sensors.

22. A method according to claim 17
wherein the plurality of in vivo sensors includes a sensor to monitor optical clarity of an image received by a laparoscopic lens located within the operative space.

23. A method according to claim 17
wherein the preprogrammed rules compare sensed environment conditions to specified upper and lower thresholds and generate control commands to maintain the sensed conditions within a range bounded by the thresholds.

24. A method according to claim 17
wherein the preprogrammed rules compare one sensed environmental condition to another sensed environmental condition and generate control commands to maintain a prescribed balance among different sensed environmental conditions.

25. A method according to claim 17
wherein the pre-programmed rules of the master controller derive closed loop control commands for airflow devices having different functions affecting the environmental conditions within the operative space.

26. A method according to claim 17
wherein the preprogrammed rules derive control commands that are proportional to sensed absolute deviations from control threshold(s).

27. A method according to claim 17
wherein the preprogrammed rules derive control commands that are based upon changes in sensed deviations from control threshold(s) over time.

28. A method according to claim 17
wherein the preprogrammed rules derive control commands that are based upon a rate of changes in sensed deviations over time.

29. A method according to claim 17
wherein the preprogrammed rules compare changes in sensed deviations from control threshold(s) over time in one sensed environmental condition to changes in sensed deviations from control threshold(s) over time in another sensed environmental condition and to generate control commands that maintain a prescribed balance among the different sensed environmental conditions.

30. A method according to claim 17
wherein the pre-programmed rules take into account, at least in part, the physical dimensions and/or properties, and/or orientation of insufflator tubing that delivers the pressurized CO2.
